(19) United States
(12) Patent Application Publication
(10) Pub. No.: US 2009/0216175 A1
(43) Pub. Date: Aug. 27, 2009

(54) Transdermal Administration Device and Method of Controlling the Same

(75) Inventors: Takehiko Matsumura, Shibuya-ku (JP); Mizuo Nakayama, Shibuya-ku (JP); Hidero Akiyama, Shibuya-ku (JP); Akihiko Matsumura, Shibuya-ku (JP); Kiyoshi Kanamura, Hachiouji-shi (JP); Akihiko Tanioka, Ohota-ku (JP)

Correspondence Address: SEED INTELLECTUAL PROPERTY LAW GROUP PLLC, 701 FIFTH AVE, SUITE 5400, SEATTLE, WA, 98104, US

(73) Assignee: TRANSCU LTD., Singapore (SG)

(21) Appl. No.: 11/917,733

(22) PCT Filed: Aug. 4, 2006
(86) PCT No.: PCT/JP2006/315530

§ 371 Date: Feb. 23, 2009

(30) Foreign Application Priority Data
Aug. 5, 2005 (JP) 2005-227377

(51) Int. Cl. A61N 1/30

(52) U.S. Cl. 604/20

(57) ABSTRACT

To provide a transdermal administration device capable of increasing the speed at which a drug is transferred into a skin and the amount of the drug to be transferred into the skin. A transdermal administration device is constituted by: an electrode supplied with a voltage of a first conductivity type; an electrolyte solution holding portion holding an electrolyte solution energized by the electrode; and a bipolar membrane that is placed on the front surface side of the electrolyte solution holding portion, and is composed of a first ion exchange membrane that selectively passes an ion of the first conductivity type and a second ion exchange membrane that selectively passes an ion of a second conductivity type.

### TECHNICAL FIELD

The present invention relates to a transdermal administration device capable of promoting the administration of a drug from the skin of an organism and a method of controlling the same.

### BACKGROUND ART

A transdermal administration method of causing a drug applied to a skin to permeate into the skin has been known from long ago. In recent years, attempts have been made to transdermally administer various drugs that have not been conventionally considered to be objects of transdermal administration.

In particular, a vaccine or an adjuvant must be delivered to a surface layer of the skin where antigen-providing cells such as Langerhans cells are mainly present, so transdermal administration has been considered to be a promising candidate of a method of administering a vaccine or an adjuvant capable of replacing intradermal injection presenting problems such as technical difficulty.

However, a sebum membrane or corneum layer with which the skin of an organism is covered serves as a barrier against an external substance, so kinds of drugs each of which can be transdermally administered in an effective dose within a time period acceptable for the administration of the drug are limited. Various vaccines and adjuvants can be hardly transferred into a skin merely by causing them to be present on the skin because each of them has a large molecular weight.

A speed of transfer into the skin can be increased to some extent by peeling or removing a part or entirety of the corneum layer. However, such treatment is not preferable because it results in losses of safety and simplicity inherent to transdermal administration. Even after such treatment has been performed, a vaccine or an adjuvant must be administered over a long time period (2 to 5 hours or longer) in such a manner that an effective dose of the vaccine or adjuvant enough to express an antigen-antibody reaction or an immunostimulating action is transferred into a skin.

Meanwhile, each of the vaccine and the adjuvant is generally an amphoteric electrolyte, and is positively or negatively charged depending on the pH value of the solution of each of them. Therefore, potential methods for the administration of each of them include administration by means of iontophoresis. In many cases, however, significant increasing effects of the application of a voltage on an administration speed and a dose cannot be obtained because of, for example, a small charge amount per molecular weight.

[Patent Document 1] WO 00/44438

### DISCLOSURE OF THE INVENTION

[Problems to be solved by the Invention]

The present invention has been made in view of the above problems, and an object of the present invention is to provide a transdermal administration device capable of increasing the speed at which a drug is transferred into a skin in the transdermal administration of the drug and a method of controlling the same.

Another object of the present invention is to provide a transdermal administration device capable of increasing the speed at which a drug at least a part of which dissociates to plus or minus drug ions in a drug solution is transferred into a skin and a method of controlling the same.

Another object of the present invention is to provide a transdermal administration device capable of administering a drug such as a vaccine or an adjuvant that cannot have been administered by means of a conventional transdermal administration method, a drug that must have been subjected to a treatment such as the removal of a corneum layer for the administration of an effective dose thereof, or a drug that has required a long time period for the administration of an effective dose thereof without any treatment such as the removal of a corneum layer or in an increased amount for a short time period as compared to a conventional transdermal administration method.

[Means for solving the Problems]

According to one aspect of the present invention, there is provided a transdermal administration device characterized by including: an electrode supplied with a voltage of a first conductivity type; an electrolyte solution holding portion holding an electrolyte solution energized by the electrode; and a bipolar membrane that is placed on the front surface side (skin side) of the electrolyte solution holding portion, and is composed of a first ion exchange membrane that selectively passes an ion of the first conductivity type and a second ion exchange membrane that selectively passes an ion of a second conductivity type.

The transdermal administration device according to the present invention is used in such a manner that, in a state where the front surface side of the bipolar membrane is brought into contact with a drug solution whose drug component dissociates to a plus or minus drug ion, the drug solution being placed on a skin, a voltage of a conductivity type opposite to that of the drug ion is applied to an electrode, and the device is intended for promoting the transdermal administration of a drug.

The mechanism with which the transdermal administration of a drug is promoted by the present invention is considered to be as follows.

The skin of an organism typically has a pH value of about 5 to 6, and shows weak cation selectivity in this state. It is known that increasing the pH value (to about 8 to 9) increases the cation selectivity of the skin, while reducing the pH value (to about 2 to 4) causes the skin to show anion selectivity.

Meanwhile, a drug in a drug solution dissociates to drug ions at a certain degree of dissociation, and the remainder of the drug is probably present in the drug solution in a drug molecule state.

For example, in the case of an anionic drug whose drug component dissociates to minus drug ions, the minus drug ions and neutral drug molecules are present in a drug solution. However, when a skin is provided with cation selectivity, the minus drug ions cannot be transferred into the skin, and only the neutral drug molecules can be transferred into the skin.

Therefore, when the pH of a drug solution is equal to or higher than a certain value (for example, the pH is 5 or more), the number of drug molecules that can be transferred into a skin reduces and the administration speed of a drug reduces with increasing degree of dissociation of the drug to drug ions.

The same holds true for a cationic drug whose drug component dissociates to plus drug ions. When the pH of a drug solution is equal to or lower than a certain value (for example, the pH is 4 or less), the number of drug molecules that can be transferred into a skin reduces and the administration speed of a drug reduces with increasing degree of dissociation of the drug to drug ions.

The transdermal administration device of the present invention adjusts the ion selectivity of a skin by increasing or reducing the pH value of the skin through the transfer of an H⁺ ion or an OH⁻ ion to be supplied to the front surface side (skin side) of the bipolar membrane to the skin by means of a plus or minus voltage applied to an electrode, thereby increasing the administration speed or dose of the drug.

That is, when an anionic drug is transdermally administered, a plus voltage is applied to the electrode to cause the electrolysis of water in the bipolar membrane, and an H⁺ ion generated by the electrolysis can be supplied to the front surface side of the bipolar membrane. The H⁺ ion is transferred to a skin by the action of the plus voltage applied to the electrode, so the pH value of the skin can be reduced, and the skin can be provided with anion selectivity. Accordingly, not only a drug molecule but also a minus drug ion in a drug solution can be transferred into the skin, whereby the administration speed or dose of the drug increases.

Similarly, when a cationic drug is transdermally administered, a minus voltage is applied to the electrode to cause an OH⁻ ion, which is to be supplied to the front surface of the bipolar membrane, to be transferred to a skin, to thereby increase the pH value of the skin. As a result, the skin can be provided with cation selectivity. Accordingly, not only a drug molecule but also a plus drug ion in a drug solution can be transferred into the skin, whereby the administration speed or dose of the drug increases.

Although not quantitatively confirmed, in the present invention, an electrophoresis flow is generated by the electrophoresis of an H⁺ ion or an OH⁻ ion to be supplied from the bipolar membrane toward a skin by a voltage to be applied to the electrode. The electrophoresis flow is expected to achieve an accelerating effect on the transfer of a drug ion and a drug molecule in a drug solution into a skin (electroosmosis) together.

The first ion exchange membrane in the present invention is an ion exchange membrane selectively passing an ion of the first conductivity type, and an ion exchange membrane into which an ion exchange group of the first conductivity type (an exchange group using an ion of the first conductivity type as a counter ion) can be used therefore. An arbitrary cation exchange membrane or anion exchange membrane available in the market can be used for the first ion exchange membrane of the present invention. An ion exchange membrane of a type in which a porous film having cavities a part or whole of which are filled with an ion exchange resin into which an ion exchange group of the first conductivity type is introduced can be particularly preferably used therefore.

The term “selectively passing an ion of the first conductivity type” in the foregoing refers to a state where an ion of the first conductivity type can more easily pass than an ion of the second conductivity type, and does not necessarily refer to a state where no ion of the second conductivity type can pass or a state where no restrictions are imposed on the passage of an ion of the first conductivity type.

The second ion exchange membrane in the present invention is an ion exchange membrane selectively passing an ion of the second conductivity type, and an ion exchange membrane into which an ion exchange group of the second conductivity type (an exchange group using an ion of the second conductivity type as a counter ion) can be used therefore. An arbitrary cation exchange membrane or anion exchange membrane available in the market can be used for the second ion exchange membrane of the present invention. An ion exchange membrane of a type in which a porous film having cavities a part or whole of which are filled with an ion exchange resin into which an ion exchange group of the second conductivity type is introduced can be particularly preferably used therefore.

The term “selectively passing anion of the second conductivity type” in the foregoing refers to a state where an ion of the second conductivity type can more easily pass than an ion of the first conductivity type, and does not necessarily refer to a state where no ion of the first conductivity type can pass or a state where no restrictions are imposed on the passage of an ion of the second conductivity type.

The bipolar membrane of the present invention is constituted by the first ion exchange membrane and the second ion exchange membrane described above. However, both the membranes are not necessarily needed to be integrated with each other through joining or the like. The bipolar membrane can be constituted by merely arranging (laminating) both the membranes; provided, however, that, both the membranes are preferably arranged so that they are in close contact with each other, that is, so that neither air nor any other layer is interposed between them for facilitating the occurrence of the electrolysis of water at the bipolar membrane.

The electrolyte solution in the present invention serves to establish conduction between the electrode and the bipolar membrane and to supply water to the bipolar membrane. An electrolyte solution prepared by dissolving an arbitrary electrolyte can be used. In the present invention, conduction can be secured by the movement of an H⁺ ion or an OH⁻ ion generated by the electrolysis of water at the bipolar membrane to the side of the electrode. Therefore, the electrolyte solution holding portion of the present invention can hold pure water containing no electrolyte as an electrolyte solution. It should be noted that water can be supplied to the bipolar membrane also from the drug solution.

The term “electrolyte solution holding portion” as used herein refers to a portion holding the electrolyte solution in the transdermal administration device, and the portion is not necessarily needed to be formed of a tangible member such as a container. In addition, the electrolyte solution holding portion may hold the electrolyte solution in a liquid state, or may hold the electrolyte solution after a carrier such as a gauze, cotton, filter paper, or a gel has been impregnated with the electrolyte solution.

As described above, the transdermal administration device of the present invention promotes the transfer of a drug into a skin through the application of a voltage of the first conductivity type to the electrode in a state where the front surface side of the bipolar membrane is brought into contact with a drug solution placed on the skin. The drug solution can be placed on the skin by applying the drug solution to the skin. Alternatively, the drug solution can be placed by mounting a carrier such as a gauze, cotton, filter paper, or a gel impregnated with the drug solution on the skin.

The term “voltage of the first conductivity type” in the foregoing means a plus or minus voltage. Whether a plus voltage or a minus voltage is applied to the electrode in the transdermal administration device of the present invention is determined by the conductivity type of a drug ion in a drug solution. A plus voltage is applied to the electrode when the drug ion is a minus ion, while a minus voltage is applied to the electrode when the drug ion is a plus ion. When a drug is an amphoteric electrolyte such as a protein or a peptide, the conductivity type of the drug ion varies depending on the pH value of the drug solution. In such case, the polarity of a voltage to be applied to the electrode is determined depending on the pH value of the drug solution.

In the present invention, a voltage is not necessarily needed to be applied to the electrode over the entire time period in which a drug is transdermally administered.

That is, in the present invention, anion selectivity or cation selectivity is imparted by causing an H⁺ ion or an OH⁻ ion generated at the bipolar membrane to transfer to a skin through the application of a voltage to the electrode. The skin to which anion selectivity or cation selectivity has been imparted holds the imparted ion selectivity over a certain time period even when the application of a voltage to the electrode is suspended, that is, even when the transfer of an H⁺ ion or an OH⁻ ion to the skin is suspended. As a result, during the time period, increasing effects on the speed at which a drug is transferred into the skin and the amount of the drug to be transferred into the skin in the present invention are maintained even when no voltage is applied to the electrode.

Accordingly, a voltage can be intermittently applied to the electrode in the present invention. Alternatively, after necessary anion selectivity or cation selectivity has been imparted to the skin, a voltage to be applied to the electrode can be reduced.

In the transdermal administration device of the present invention, a variation in pH value of the skin can be made larger than a variation in pH value of the drug solution by controlling the profile of a voltage to be applied to the electrode.

Therefore, when one wishes to maintain the pH value of the drug solution at a certain value (or at a certain value or higher or lower) in consideration of a relationship with a drug effect, the profile of a voltage to be applied to the electrode is controlled, whereby the pH value of the drug solution can be made to remain nearly unchanged while a change in pH value enough to impart necessary ion selectivity to the skin is given.

The term “drug” as used herein refers to a substance which may be or may not be prepared, which has a certain pharmacological action, and which is administered to an organism for purposes including the therapy, recovery, and prevention of a disease, the maintenance and promotion of the health, the maintenance and promotion of beautification, and a weight loss. The term “drug” as used herein includes a vaccine, allergen, or adjuvant expressing an antigen-antibody reaction or an immunostimulating action. Therefore, the term “pharmacological action” includes an antigen-antibody reaction and an immunostimulating action.

The term “drug ion” as used herein refers to an ion which is produced by the dissociation of a drug to ions and which is responsible for a pharmacological action. The dissociation of the drug to a drug ion may occur as a result of the dissolution of the drug into a solvent such as water, an acid, or an alkali, or may occur as a result of, for example, the application of a voltage or the addition of an ionizing agent.

The term “drug solution” as used herein includes various states such as a drug suspended or emulsified into a solvent and the drug adjusted to be in an ointment state or a paste state in addition to a liquid-like solution prepared by dissolving the drug as long as at least a part of the drug dissociates to drug ions in a solution. In addition, the drug solution may be used in a liquid, suspension, emulsion, ointment, or paste state, or a carrier such as a gauze, filter paper, or a gel may be impregnated with such drug solution before the solution is used.

The term “first conductivity type” as used herein refers to plus or minus electrical polarity, and the term “second conductivity type” as used herein refers to the electrical polarity (minus or plus) opposite to the first conductivity type.

The first ion exchange membrane in the present invention is preferably placed on the front surface side of the second ion exchange membrane. In this case, the electrolysis of water at an interface between the first ion exchange membrane and the second ion exchange membrane can be caused to efficiently occur.

The transport number of each of the first ion exchange membrane and the second ion exchange membrane in the present invention is preferably 0.95 or more, or particularly preferably 0.98 or more. In this case, the electrolysis of water at the interface between the first ion exchange membrane and the second ion exchange membrane can be caused to efficiently occur.

The transport number of the first/second ion exchange membrane can be controlled depending on, for example, the kind and amount of an ion exchange resin in the first/second ion exchange membrane and the kind and amount of an ion exchange group to be introduced to the ion exchange resin.

The transport number of the first ion exchange membrane in the foregoing is defined as a ratio of charge conveyed by the transfer of an ion of the first conductivity type in the electrolyte solution to the side of the drug solution to the total charge conveyed through the first ion exchange membrane when a voltage of the first conductivity type is applied to the side of the electrolyte solution in a state where only the first ion exchange membrane is placed between the electrolyte solution and the drug solution. The transport number of the second ion exchange membrane is defined as a ratio of charge conveyed by the transfer of an ion of the second conductivity type in the drug solution to the side of the electrolyte solution to the total charge conveyed through the second ion exchange membrane when a voltage of the first conductivity type is applied to the side of the electrolyte solution in a state where only the second ion exchange membrane is placed between the electrolyte solution and the drug solution.

The transdermal administration device of the present invention can further include, on the front surface side of the bipolar membrane, a drug solution holding portion holding a drug solution containing a drug whose drug component dissociates to drug ions of the second conductivity type. In this case, the convenience of the administration of a drug can be enhanced.

The transdermal administration device of the present invention can hold at least one or more kinds of adjuvants in the drug solution holding portion. With the transdermal administration device, an adjuvant can be administered without the peeling or removal of a corneum layer on a skin surface or in a shorter time period than that in the case of conventional transdermal administration. Examples of an adjuvant that can be preferably used in the present invention include LT, CT, CpG, ETA, and PT.

The transdermal administration device of the present invention can hold at least one or more kinds of vaccines in the drug solution holding portion. With the transdermal administration device, a vaccine can be administered without the peeling or removal of a corneum layer on a skin surface or in a shorter time period than that in the case of conventional transdermal administration. Examples of a vaccine that can be preferably used in the present invention include vaccines for influenza, a cancer, and hepatitis (A type and B type).

The transdermal administration device of the present invention can further include control means for intermittently applying a voltage to the electrode. In this case, the simplicity of the transdermal administration of a drug can be enhanced.

The transdermal administration device of the present invention can further include: pH measuring means for measuring the pH value of a skin; and control means for controlling a voltage to be applied to the electrode in accordance with a pH value measured by the pH measuring means. In this case, the pH value of the skin can be maintained at an appropriate value. As a result, an increase in administration speed or dose of a drug can be achieved. At the same time, the stability and safety of the administration of the drug can be additionally improved.

The transdermal administration device of the present invention can further include a second electrode as a counter electrode of the electrode supplied with a voltage of the first conductivity type.

According to another aspect of the present invention, there is provided a method of controlling a transdermal administration device, characterized by including: bringing one surface of a bipolar membrane composed of a first ion exchange membrane that selectively passes an ion of a first conductivity type and a second ion exchange membrane that selectively passes an ion of a second conductivity type into contact with a drug solution whose drug component dissociates to drug ions of the second conductivity type, the drug solution being placed on the skin of an organism; bringing another surface of the bipolar membrane into contact with an electrolyte solution; and applying a voltage of the first conductivity type from the side of the electrolyte solution to promote the transfer of a drug in the drug solution into the organism.

In the present invention, an H⁺ ion or an OH⁻ ion supplied from the bipolar membrane is transferred to the skin of an organism by a voltage of the first conductivity type applied from the side of the electrolyte solution, whereby anion selectivity or cation selectivity is imparted to the skin of the organism. As a result, the speed at which a drug is transferred into the skin or the amount of the drug to be transferred into the skin can be increased.

In this case, the voltage can be applied intermittently. Alternatively, the voltage to be applied from the side of the electrolyte solution can be controlled on the basis of the pH value of a skin surface.

### [Best Mode for Carrying Out the Invention]

Hereinafter, an embodiment of the present invention will be described with reference to the drawings.

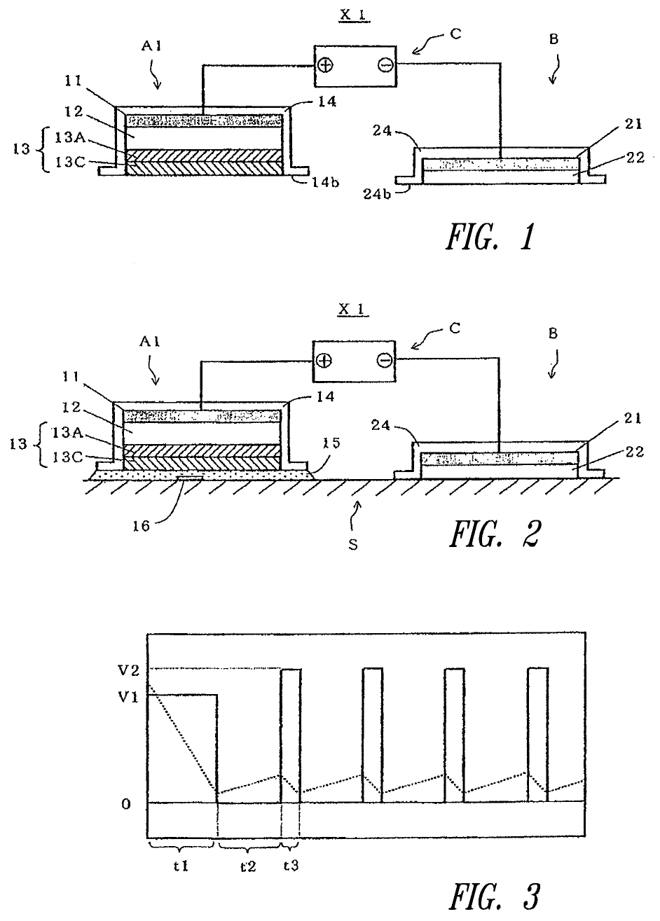

FIG. 1 is a schematic sectional view showing the constitution of a transdermal administration device according to the present invention.

In the following description, for convenience of description, a transdermal administration device for administering a drug whose drug component dissociates to minus drug ions (for example, ascorbic acids as a vitamin agent) is exemplified. In the case of a transdermal administration device for administering a drug whose drug component dissociates to plus drug ions (for example, lidocaine hydrochloride as an anesthetic drug or morphine hydrochloride as an anesthetic drug), the polarity (plus or minus) of an electric power source terminal to be connected to each electrode member and the polarity (a cation exchange membrane or an anion exchange membrane) of each ion exchange membrane in the following description are reversed. In addition, in the case of a transdermal administration device for administering a drug composed of an amphoteric electrolyte whose drug ions each change its polarity depending on pH, which type of transdermal administration device is used is selected depending on pH.

As shown in the figure, a transdermal administration device X1 of the present invention includes a working assembly A1, a non-working assembly B, and an electric power source C as main components (members).

The working assembly A1 includes an electrode member 11 connected to the plus pole of the electric power source C, an electrolyte solution holding portion 12 holding an electrolyte solution kept so as to be in contact with the electrode member 11, and a bipolar membrane 13 composed of an anion exchange membrane 13A and a cation exchange membrane 13C, the bipolar membrane 13 being placed on the front surface side (skin side) of the electrolyte solution holding portion 12. The entire working assembly A1 is housed in a cover or container 14.

Meanwhile, the non-working assembly B includes an electrode member 21 connected to the minus pole of the electric power source C and an electrolyte solution holding portion 22 holding an electrolyte solution kept so as to be in contact with the electrode member 21. The entire non-working assembly B is housed in a cover or container 24.

In the transdermal administration device X1, an arbitrary conductive material can be used for each of the electrode members 11 and 21 without any particular limitation. An active electrode made of silver/silver chloride or the like is preferably used for preventing a reduction in energization property due to the generation of a gas as a result of the electrolysis of water at each of the electrode members 11 and 21.

An electrolyte solution prepared by dissolving an arbitrary electrolyte can be used for each of the electrolyte solution holding portions 12 and 22 for securing energization property with respect to the bipolar membrane 13 or a skin. The generation of a gas at each of the electrode members 11 and 21 described above can be prevented by using an electrolyte solution prepared by dissolving an electrolyte having an oxidation-reduction potential lower than that of water. In such case, there is no need to use an active electrode for each of the electrode members 11 and 21.

Each of the electrolyte solution holding portions 12 and 22 may hold the electrolyte solution in a liquid state, or may hold the electrolyte solution after a carrier such as a gauze, cotton, filter paper, or an acrylic or polyurethane-based gel has been impregnated with the electrolyte solution.

Known examples of an ion exchange membrane include various ion exchange membranes such as (1) a heterogeneous ion exchange membrane obtained by: dispersing an ion exchange resin in a binder polymer; and forming the resultant into a film through, for example, molding under heat and (2) a homogeneous ion exchange membrane obtained by: impregnating and filling a base material such as cloth, a net, or a porous film composed of a polyolefin resin, a fluorine-based resin, or a polyamide resin with a solution prepared by dissolving a composition composed of a monomer, cross-linkable monomer, polymerization initiator, or the like into which an ion exchange group can be introduced or a resin having a functional group into which an ion exchange group can be introduced into a solvent; subjecting the resultant to polymerization or solvent removal; and subjecting the resultant to a treatment for introducing an ion exchange group as well as an ion exchange resin formed into a membrane shape. Any one of those arbitrary ion exchange membranes can be used for each of the anion exchange membrane 13A and the cation exchange membrane 13C without any particular limitation.

Examples of an anion exchange group to be introduced into the anion exchange membrane 13A include a primary amino group, a secondary amino group, a tertiary amino group, a quaternary ammonium group, a pyridyl group, an imidazole group, a quaternary pyridinium group, and a quaternary imidazolium group. The transport number of an anion exchange membrane can be controlled depending on the kind of an anion exchange group to be introduced. For example, the use of a quaternary ammonium group or a quaternary pyridinium group as a strong basic group provides an anion exchange membrane having a high transport number.

Examples of a cation exchange group to be introduced into the cation exchange membrane 13C include a sulfonic group, a carboxylic group, and a phosphoric group. The transport number of a cation exchange membrane can be controlled depending on the kind of a cation exchange group to be introduced. For example, the use of a sulfonic group as a strong acidic group provides a cation exchange membrane having a high transport number.

Known examples of a treatment for introducing an anion exchange group include various approaches such as amination and alkylation. Known examples of a treatment for introducing a cation exchange group include various approaches such as sulfonation, chlorosulfonation, phosphonation, and hydrolysis. The transport number of an ion exchange membrane can be adjusted by adjusting conditions under which a treatment for introducing an ion exchange group is performed.

In addition, the transport number of an ion exchange membrane can be adjusted depending on, for example, the amount of an ion exchange resin in the ion exchange membrane and the pore size and pore ratio of the membrane. For example, in the case of an ion exchange membrane of a type in which a porous film is filled with an ion exchange resin, an ion exchange membrane obtained by filling a porous film with an ion exchange resin at a filling ratio of preferably 5 to 95 mass %, more preferably 10 to 90 mass %, or particularly preferably 20 to 60 mass % can be used, the porous film having formed thereon a large number of small pores having a mean pore size of preferably 0.005 to 5.0 μm, more preferably 0.01 to 2.0 μm, or most preferably 0.02 to 0.2 μm (a mean flow pore size measured in conformance with the bubble point method (JIS K3832-1990)) at a porosity of preferably 20 to 95%, more preferably 30 to 90%, or most preferably 30 to 60% and having a thickness of preferably 5 to 140 μm, more preferably 10 to 120 μm, or most preferably 15 to 55 μm. The transport number of the ion exchange membrane can be adjusted depending also on the mean pore size and porosity of the small pores of the porous film and the filling ratio of the ion exchange resin.

Specifically, an ion exchange membrane into which an anion exchange group is introduced such as a NEOSEPTA (AM-1, AM-3, AMX, AHA, ACH, or ACS) manufactured by Tokuyama Co., Ltd can be used for the anion exchange membrane 13A. An ion exchange membrane into which a cation exchange group is introduced such as a NEOSEPTA (CM-1, CM-2, CMX, CMS, or CMB) manufactured by Tokuyama Co., Ltd can be used for the cation exchange membrane 13C.

A membrane having as high a transport number as possible is preferably used for each of the anion exchange membrane 13A and the cation exchange membrane 13C in such a manner that electrolysis occurs at an interface between the anion exchange membrane 13A and the cation exchange membrane 13C at as low an applied voltage as possible. The transport number of each of the anion exchange membrane 13A and the cation exchange membrane 13C is in the range of preferably 0.95 or more, or particularly preferably 0.98 or more.

The cover or container 14 or 24 can be formed of an arbitrary material such as a plastic or a metal film which is capable of preventing: the evaporation or leakage of water from each of the electrolyte solution holding portions 12 and 22; or the mixing of foreign matter from the outside, and which has a strength of such magnitude that the material does not break during handling. An adhesive layer for improving adhesiveness to a skin or a drug solution layer can be arranged on a bottom portion 14b or 24b of the cover or container 14 or 24.

A liner for preventing: the evaporation or leakage of water from each of the electrolyte solution holding portions 12 and 22; or the mixing of foreign matter from the outside during storage of the transdermal administration device X1 can be stuck to the front surface side of the bipolar membrane 13 and/or the front surface side of the electrolyte solution holding portion 22.

A battery, a constant voltage device, a constant current device, a constant voltage/current device, a variable voltage electric power source, or the like can be used as the electric power source C.

FIG. 2 is an explanatory view showing how the transdermal administration device X1 is used.

The transdermal administration device X1 is used in such a manner that a plus voltage and a minus voltage are applied to the electrode members 11 and 12, respectively, in a state where a drug solution layer 15 placed on a skin S is brought into contact with the front surface side of the bipolar membrane 13 (the front surface side of the cation exchange membrane 13C) and the electrolyte solution holding portion 22 is brought into contact with another site of the skin S as shown in the figure. In the figure, reference numeral 16 denotes a pH sensor for monitoring a pH value on the skin S during administration of a drug. It is needless to say that there is no need to use the pH sensor 16 when there is no need to monitor a pH value during administration of a drug.

The drug solution layer 15 contains a drug whose drug component dissociates to minus drug ions. The drug solution layer 15 can be formed by applying a drug solution in the state of a liquid or the like to the skin S. Alternatively, a carrier such as a gauze, cotton, filter paper, or an acrylic or polyurethane-based gel impregnated with the drug solution may be placed on the skin S.

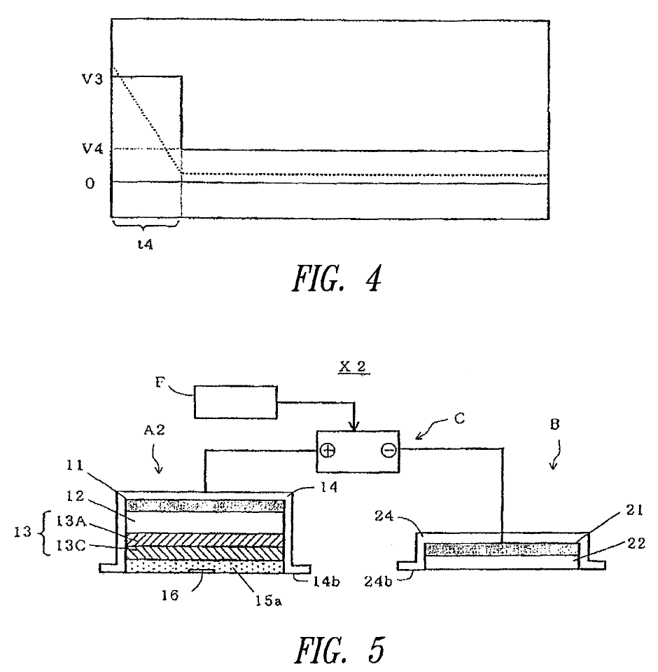

FIGS. 3 and 4 each show the profile of a voltage applied to the electrode member 11 during administration of a drug (solid line) and the transition of a pH value detected by the pH sensor 16 (broken line).

In the profile of FIG. 3, a plus voltage V1 is continuously applied over a predetermined time period t1 in a first phase.

At this time, an H⁺ ion generated by the electrolysis of water in the bipolar membrane 13 is supplied to the front surface side of the bipolar membrane 13, and the ion is transferred into the skin S by the action of the plus voltage V1. As a result, the pH value of the skin S reduces, and anion selectivity can be imparted to the skin S. Therefore, not only a drug molecule but also a drug ion as a minus ion in the drug solution layer 15 can be transferred into the skin S.

In the first phase, the action with which a drug ion is attracted to the side of the electrode member 11 by the plus voltage V1 and the action (electroosmosis) with which an electrophoresis flow generated by the movement of an H⁺ ion to the side of the skin S causes the drug ion to flow to the side of the skin S compete with each other. In any case, the transfer of a certain amount of drug ions into the skin is expected to occur. In addition, the amount of drug molecules to be transferred into the skin also increases owing to the electroosmosis generated by the electrophoresis flow. Therefore, as compared to the case where the transdermal administration device X1 is not used, the amount of drug ions to be transferred into the skin and the amount of drug molecules to be transferred into the skin owing to the electroosmosis purely increase, so the administration speed or dose of the drug can be surely increased.

After the first phase, the suspension of the application of a voltage for a predetermined time period t2 (a second phase) and the application of a plus voltage V2 identical to or different from the plus voltage V1 for a predetermined time period t3 (a third phase) are repeated.

In the second phase, the skin S gradually increases its pH value so that its pH value returns to the original pH value over a certain relaxation time. The anion selectivity of the skin S can be maintained throughout the second and third phases by applying the voltage V2 in the third phase before the anion selectivity of the skin S is lost.

In the second phase, in addition to the transfer of a drug molecule into the skin due to scattering, the transfer of a drug ion into the skin due to scattering occurs. Therefore, as compared to the case where the transdermal administration device X1 is not used, the administration speed or dose of the drug increases. In the third phase, the administration speed or dose of the drug increases via the same mechanism as that described above with respect to the first phase.

In the profile of FIG. 4, in a fourth phase, a predetermined plus voltage V3 is continuously applied over a predetermined time period t4. After that, a predetermined plus voltage V4 lower than V3 is continuously applied in the second phase.

In the fourth phase in FIG. 4, the pH value of the skin S reduces in the same manner as in the first phase in FIG. 3, whereby anion selectivity is imparted. In a fifth phase, an increase in pH value due to the relaxation of the skin S and a moderate reduction in pH value due to the plus voltage V4 compete with each other. As a result, the pH value of the skin is kept constant.

In the fourth phase, the administration speed or dose of the drug increases via the same mechanism as that of the first phase in FIG. 3. In the fifth phase, each of the action with which a drug ion is attracted to the side of the electrode member 11 by the plus voltage V4 and the action (electroosmosis) with which the electrophoresis flow of an H⁺ ion causes the drug ion and the drug molecule to flow to the side of the skin S is smaller than that in the fourth phase. However, the administration speed or dose of the drug increases via the same mechanism as that of the fourth phase.

It should be noted that, in any one of the above profiles, the time periods t1 to t3 and the voltages V1 to V4 can be appropriately adjusted depending on, for example, a site of the skin S, the kind of the drug, and the pH value and amount (the thickness of the drug layer 16) of the drug solution.

FIG. 5 is an explanatory view showing a transdermal administration device X2 according to another embodiment of the present invention.

The transdermal administration device X2 is different from the transdermal administration device X1 except that the device X2 includes: a drug solution holding portion 15a on the front surface side of the bipolar membrane 13; the pH sensor 16 on the front surface side of the drug solution holding portion 15a; and a control circuit F connected to the pH sensor 16 by means of wiring (not shown), the circuit being intended for controlling the output of the electric power source C on the basis of a value detected by the sensor. The other constitutions of the device X2 are the same as those of the transdermal administration device X1.

The drug solution holding portion 15a of the transdermal administration device X2 holds a drug solution containing a drug whose drug component dissociates to minus drug ions. The drug solution holding portion 15a may hold the drug solution in the state of a liquid without any change. Alternatively, the portion may be constituted by a carrier such as a gauze, cotton, filter paper, or an acrylic or polyurethane-based gel impregnated with the drug solution.

A sensor of an arbitrary type suitable for the measurement of a pH value on the surface of a skin or in the skin such as a commercially available glass electrode pH sensor or a semiconductor pH sensor using ISFET can be used for the pH sensor 16.

In the transdermal administration device X2, a plus voltage and a minus voltage are applied from the electric power source C to the electrode members 11 and 21, respectively on the basis of a signal from the control circuit F in a state where the drug solution holding portion 15a and the electrolyte solution holding portion 22 are brought into contact with the skin of an organism. As a result, the administration of a drug from the drug solution holding portion 15a into the skin is promoted.

The control circuit F can be adapted to control the output of the electric power source C in the manner shown in FIG. 3 or 4 by causing the electric power source C to output a voltage when the pH value measured by the pH sensor 16 is equal to or larger than a predetermined value and by suspending or reducing the voltage outputted from the electric power source C when the pH value is equal to or smaller than the predetermined value.

In addition, the control circuit F can control the output of the electric power source C in the manner shown in FIG. 3 or 4 on the basis of only the time period elapsed from the initiation of the administration of a drug in accordance with a predetermined program. In this case, the transdermal administration device X2 is not requested to include the pH sensor 16.

The present invention has been described above byway of several embodiments. However, the present invention is not limited to those embodiments, and can be variously altered within the scope of claims.

For example, in each of the above embodiments, description has been given of the case where the non-working assembly B includes the electrolyte solution holding portion 22 or the case 24. Alternatively, the non-working assembly B may have any other arbitrary constitution as long as it includes a member capable of applying a voltage opposite to that of the electrode member 11 (or the earth) to the skin of an organism. For example, the non-working assembly B may include neither the electrolyte solution holding portion 22 nor the case 25.

Alternatively, the transdermal administration device itself may not include the non-working assembly B. For example, the transfer of a drug into an organism can be promoted by applying a voltage to the working assembly in a state where a part of the organism is brought into contact with a member to serve as the earth while the working assembly is brought into contact with the skin of the organism or the drug solution layer placed on the skin of the organism.

Alternatively, the non-working assembly B can be constituted by: an electrode member; an electrolyte solution holding portion placed on the front surface side of the electrode member; an ion exchange membrane that selectively passes an ion of the first conductivity type, the ion exchange membrane being placed on the front surface side of the electrolyte solution holding portion; an electrolyte solution holding portion placed on the front surface side of the ion exchange membrane; and an ion exchange membrane that selectively passes an ion of the second conductivity type, the ion exchange membrane being placed on the front surface side of the electrolyte solution holding portion. With such constitution, a pH value on a skin surface upon energization can be stabilized.

In addition, in any one of the above cases, as in the case of the transdermal administration device shown as an embodiment, the basic effect of the present invention, that is, an increasing effect on the speed at which a drug is transferred to an organism or the amount of the drug to be administered to the organism can be achieved by imparting appropriate ion selectivity to a skin. Any one of the cases is included in the scope of the present invention.

The voltage profiles shown in the embodiments are examples. Any other voltage profile with which the ion selectivity of a skin can be appropriately controlled can be used to transdermally administer a drug. The present invention is not limited by the voltage profiles in the embodiments.

Furthermore, in each of the above embodiments, the case has been described where the working assembly, the non-working assembly, the electric power source, the control circuit, and the like are constituted separately. It is also possible that a part or whole of those elements are incorporated in a single casing or an entire device incorporating them is formed in a sheet shape or a patch shape, whereby the handle ability thereof is enhanced, and such transdermal administration device is also included in the scope of the present invention.

### BRIEF DESCRIPTION OF THE DRAWINGS

[FIG. 1] An explanatory view showing the constitution of a transdermal administration device according to an embodiment of the present invention.

[FIG. 2] An explanatory view showing how the transdermal administration device according to the embodiment of the present invention is used.

[FIG. 3] An explanatory view showing an exemplary voltage profile to be applied to an electrode of the transdermal administration device of the present invention.

[FIG. 4] An explanatory view showing an exemplary voltage profile to be applied to the electrode of the transdermal administration device of the present invention.

[FIG. 5] An explanatory view showing the constitution of a transdermal administration device according to another embodiment of the present invention.

### DESCRIPTION OF SYMBOLS

X1, X2 TRANSDERMAL ADMINISTRATION DEVICE

A1, A2 WORKING ASSEMBLY

11 ELECTRODE MEMBER

12 ELECTROLYTE SOLUTION HOLDING PORTION

13 BIPOLAR MEMBRANE

13A ANION EXCHANGE MEMBRANE

13C CATION EXCHANGE MEMBRANE

14 CONTAINER

15 DRUG SOLUTION LAYER

15a DRUG SOLUTION HOLDING PORTION

16 pH SENSOR

B NON-WORKING ASSEMBLY

21 ELECTRODE MEMBER

22 ELECTROLYTE SOLUTION HOLDING PORTION

24 CONTAINER

C ELECTRIC POWER SOURCE

1-12. (canceled)

13. A transdermal administration device, comprising:
an electrode operable to receive a voltage of a first charge type;
an electrolyte solution holding portion; and
a bipolar membrane located on a front surface side of the electrolyte solution holding portion, the bipolar membrane comprising a first ion exchange membrane that selectively passes an ion of the first charge type and a second ion exchange membrane that selectively passes an ion of a second charge type, the first ion exchange membrane having a transport number of about 0.95 or greater for the ions of the first charge type, and the second ion exchange membrane having a transport number of about 0.95 or greater for the ions of the second charge type.

14. The transdermal administration device according to claim 13 wherein the first ion exchange membrane comprises a transport number of about 0.98 or greater for the ions of the first charge type, and the second ion exchange membrane comprises a transport number of about 0.98 or greater for the ions of the second charge type.

15. The transdermal administration device according to claim 13 wherein the first ion exchange membrane is located on a front surface side of the second ion exchange membrane.

16. The transdermal administration device according to claims 13, further comprising:
a drug solution holding portion located on a front surface side of the bipolar membrane, the drug solution holding portion holding a drug solution including a drug having a medicinal effective ingredient that dissociates into a drug ion of the second charge type.

17. The transdermal administration device according to claim 16 wherein the drug solution further includes at least one kind of adjuvant.

18. The transdermal administration device according to claim 16 wherein the drug solution further includes at least one kind of vaccine.

19. The transdermal administration device according to claim 16 wherein the drug solution holding portion includes a carrier selected from the group consisting of a gauze, cotton, filter paper, an acrylic-based gel, and a polyurethane-based gel.

20. The transdermal administration device according to claim 13, further comprising:
a controller operable to intermittently apply a voltage to the electrode.

21. The transdermal administration device according to claim 13, further comprising:
a controller operable to intermittently apply a voltage of the first charge type or the second charge type to the electrode.

22. The transdermal administration device according to claims 13, further comprising:
a pH sensor operable to determining or monitor a pH value of a skin surface; and
a controller operable to control a voltage applied to the electrode in accordance with a pH value determined or monitored by the pH sensor.

23. The transdermal administration device according to claim 13, further comprising:
a second electrode supplied with a voltage of the second charge type.

24. A method for controlling a transdermal administration device, comprising:
contacting a skin of a living body, that has been treated with a drug solution including a medicinal effective ingredient that dissociates into a drug ion of a second charge type, with a first surface side of a bipolar membrane composed of a first ion exchange membrane that selectively passes an ion of a first charge type and a second ion exchange membrane that selectively passes an ion of the second charge type, the first ion exchange membrane having a transport number of about 0.95 or greater for the ions of the first charge type, and the second ion exchange membrane having a transport number of about 0.95 or greater for the ions of the second charge type;
providing an electrode and an electrolyte solution holding portion located on a second surface side opposite to first surface side of the bipolar membrane;
sensing a pH of the skin of a living body in contact with the first surface; and
intermittently applying a voltage of the first charge type to the electrode based on the sensed pH.

25. The method of claim 24 wherein contacting the skin with the first surface side of the bipolar membrane includes contacting the skin with the first surface side of the bipolar membrane having a transport number of about 0.98 or greater for the ions of the first charge type, and the second ion exchange membrane having a transport number of about 0.98 or greater for the ions of the second charge type.

26. The method of claim 24 wherein intermittently applying a voltage includes applying a negative or a positive voltage to maintain the sensed pH of the skin at or about a target value.

27. The method of claim 24 wherein intermittently applying a voltage includes applying a sufficient negative or a sufficient positive voltage to impart an ion selectivity to the skin.

28. A method of maintaining a pH value of a drug solution at or about a target value, while imparting a sufficient change in pH of skin of a living body contacting a transdermal administration device, during transdermal administration of the drug solution, comprising:
contacting a skin of a living body with a transdermal administration device comprising a drug solution holding portion and a bipolar membrane composed of a first ion exchange membrane that selectively passes an ion of a first charge type and a second ion exchange membrane that selectively passes an ion of the second charge type, the first ion exchange membrane having a transport number of about 0.95 or greater for the ions of the first charge type, and the second ion exchange membrane having a transport number of about 0.95 or greater for the ions of the second charge type;
sensing a pH of the skin of the living body in contact with the transdermal administration device;
sensing a pH of the drug solution holding portion; and
intermittently applying a voltage of the first charge type or the second charge type based on the sensed pH of drug solution holding portion or the sensed pH of the skin.

29. The method of claim 28 wherein intermittently applying a voltage includes applying the voltage of the first charge type to maintain the sensed pH of the skin at or about a target value

30. The method of claim 28 wherein intermittently applying a voltage includes applying the voltage of the first charge type or the second charge type to maintain the sensed pH of the skin at or about a target value

31. The method of claim 30 wherein intermittently applying a voltage includes applying a sufficient voltage of the first charge type to impart an ion selectivity to the skin.

32. The method of claim 30 wherein intermittently applying a voltage includes applying a sufficient voltage of the first charge type or a sufficient voltage the second charge type to impart an ion selectivity to the skin.
